SEQUENCE LISTING (9 sequences)

SEQ ID NO: 1 - DNA, 18 bp, Mycobacterium tuberculosis
ccactcgtagccgtacag

SEQ ID NO: 2 - DNA, 17 bp, Mycobacterium tuberculosis
tcgtatggcaccggaac

SEQ ID NO: 3 - DNA, 15 bp, Mycobacterium tuberculosis
tcgatgccggtggtg

SEQ ID NO: 4 - DNA, 17 bp, Mycobacterium tuberculosis
acaccgcagacgttgat

SEQ ID NO: 5 - DNA, 17 bp, Mycobacterium tuberculosis
acgctcacgtgacagac

SEQ ID NO: 6 - DNA, 20 bp, Mycobacterium tuberculosis
tgagccaattcatggaccag

SEQ ID NO: 7 - DNA, 20 bp, Mycobacterium tuberculosis
ctcgtggacataccgatttc

SEQ ID NO: 8 - DNA, 18 bp, Mycobacterium tuberculosis
aggactgaacgggatacg

SEQ ID NO: 9 - DNA, 16 bp, Mycobacterium tuberculosis
cgcggcgagatgatag

(19) United States
(12) Patent Application Publication
(10) Pub. No.: US 2015/0031576 A1
(43) Pub. Date: Jan. 29, 2015

(54) REAL TIME PCR DETECTION OF M. TUBERCULOSIS RESISTANT/SUSCEPTIBLE TO RIFAMPICIN AND/OR ISONIAZID

(71) Applicant: VELA OPERATIONS PTE. LTD., Singapore (SG)

(72) Inventor: Elian Rakhmanaliev, Singapore (SG)

(21) Appl. No.: 14/383,071

(22) PCT Filed: Mar. 6, 2013
(86) PCT No.: PCT/IB2013/051779

§ 371 Date: Sep. 4, 2014

(30) Foreign Application Priority Data
Mar. 6, 2012 (GB) 1203872.5

(51) Int. Cl. C12Q 1/68

(52) U.S. Cl. CPC C12Q 1/689; C12Q 2600/156; C12Q 2600/112
USPC 506/9; 435/6.11; 506/16

(57) ABSTRACT

The present invention relates to assays, diagnostic kits and methods for the real-time PCR detection of nucleic acids that are indicative of rifampicin and/or isoniazid susceptible and/or resistant Mycobacterium tuberculosis bacteria.

### TECHNICAL FIELD OF THE INVENTION

The present invention relates to diagnostics of M. tuberculosis strains that are susceptible or resistant to rifampicin and/or isoniazid, respectively.

### BACKGROUND

M. tuberculosis is the pathogen which is responsible for tuberculosis, one of the major causes of human infections of the respiratory tract and responsible for millions of fatalities every year. While a vaccine is available, immunity decreases after several years. Infected patients are usually treated with antibiotics, most commonly with isoniazid and rifampicin. In recent years the development of multidrug-resistant tuberculosis bacteria as well as largely drug-resistant Mycobacterium tuberculosis and totally drug-resistant tuberculosis strains have been reported. Most of the multidrug resistant tuberculosis strains are resistant to the two hitherto most effective first line tuberculosis drugs, i.e. isoniazid and rifampicin.

The development of resistant strains and a failure to treat such organisms with the first line medicaments may result in an increased spread of infections and wrong or inadequate treatment with a correspondingly larger numbers of fatalities or greater severity of diseases. Accordingly, it is important to detect the cause of a respiratory infection, in particular in cases where tuberculosis is suspected to be the reason for the infection, and that such detection method be highly sensitive, cost-efficient and rapid and preferably able to distinguish between M. tuberculosis strains that are resistant to isoniazid and rifampicin, or strains that are still susceptible to treatment with these drugs.

Accordingly, the present invention provides new assays, compositions, kits and methods for the diagnosis and detection of M. tuberculosis strains that are susceptible and/or resistant to the above-mentioned two first line antibiotics.

The present invention relates to a set of nucleic acid primers and probes, useful for simultaneous or separate detection of rifampicin and/or isoniazid susceptible and/or resistant M. tuberculosis strains in a biological sample using real-time PCR. The primers and probes of the invention are suitable for specifically detecting nucleotide polymorphisms in the rpoB gene, which are associated with sensitivity or resistance to rifampicin. Furthermore, primers and probes suitable for specifically recognizing nucleotide polymorphisms in the katG gene and in the promoter region of the inhA gene are used to identify whether or not the biological sample contains M. tuberculosis that is susceptible or resistant to isoniazid. Each of the target nucleic acids may be analyzed separately or simultaneously in a multiplex assay.

In a first embodiment, the first set of primers comprises sequences selected from SEQ ID Nos: 1 to 3 and SEQ ID Nos: 4 to 7, and preferably the first probe has a sequence selected from SEQ ID Nos: 8 to 10 or a complement thereof.

In a second embodiment, the second set of primers comprises sequences depicted in SEQ ID Nos: 11 and 12, and preferably the second probe has a sequence selected from SEQ ID Nos: 13 or 14 or a complement thereof.

In a third embodiment, the third set of primers comprises a sequence depicted in SEQ ID No: 15 and one of the sequences depicted in SEQ ID Nos: 16 to 19, and preferably the third probe has the sequence of SEQ ID No. 20 or a complement thereof.

The present invention also relates to a method for detecting nucleic acids that are indicative of rifampicin and/or isoniazid susceptible and/or resistant Mycobacterium tuberculosis bacteria in a biological sample from a patient, comprising:
a) providing a biological sample from a patient;
b) extracting nucleic acids from the biological sample;
c) carrying out a PCR with the set of nucleic acids according to the present invention; and
d) detecting amplification products for target nucleic acids of interest (e.g. rpoB, katG and/or inhA), wherein the presence of an amplification product is indicative of the presence of the M. tuberculosis in the biological sample
e) detecting polymorphisms in target nucleic acids indicative of rifampicin and/or isoniazid susceptibility/resistance using specific probes.

In an alternative method, instead of carrying a RT-PCR, the method comprises a step of reverse-transcription and a step of PCR amplification.

The present invention further concerns the use of a set of nucleic acids (primers and probes) according to the present invention for detecting nucleic acids that are indicative of rifampicin and/or isoniazid susceptible and/or resistant Mycobacterium tuberculosis bacteria. In addition, it concerns a set of nucleic acids according to the present invention for preparing a diagnostic kit useful for the above purpose. Optionally, the kit further comprises other components such as a DNA polymerase, a reverse-transcriptase, RNase inhibitors, dNTPs and a PCR and/or RT-buffers.

### Short Summary of Preferred Embodiments

Some of the preferred embodiments of the invention are depicted below:
i. A method for the detection of at least one rifampicin and/or isoniazid Mycobacterium tuberculosis bacterium in a biological sample, wherein the method comprises conducting real-time PCR.
ii. The method according to embodiment (i) further comprising isolating nucleic acids from the biological sample and optionally performing a reverse transcription step.
iii. The method according to any one of embodiments (i) or (ii), wherein primer sets selected from the sequences depicted in SEQ ID Nos: 1 and 2 and/or SEQ ID Nos: 4 and 5 and/or SEQ ID Nos: 7 and 8, or complements or homologues thereof are used.
iv. The method according to any one of embodiments (i) to (iii), further comprising using an oligonucleotide probe, provided said probes hybridize specifically to at least one amplification product obtainable with the primer sets according to any one of the preceding claims.
v. The method according to embodiment (iv), wherein the probe specifically recognizes nucleotide polymorphisms in at least one of the following genes: the rpoB gene, the katG gene and the inhA gene.
vi. The method according to any one of embodiments (i) to (v), wherein at least one probe is selected from the sequences depicted in SEQ ID Nos: 3, 6 and 9, or complements or homologues thereof.
vii. The method according to any one of the preceding embodiments, wherein the primers and or probes carry a fluorescent moiety.
viii. A method for the diagnosis of an Mycobacterium tuberculosis infection comprising performing one of the methods according to any one of the preceding embodiments.
ix. A method for monitoring the treatment of Mycobacterium tuberculosis infection, said method comprising performing the method according to embodiment (viii) before treatment with at least one drug and during and/or after treatment with said drug.
x. An assay for simultaneous detection of at least one nucleic acid of rifampicin and/or isoniazid susceptible and/or resistant Mycobacterium tuberculosis in a biological sample comprising primers specifically hybridizing to nucleic acids derived from said Mycobacterium tuberculosis, wherein said assay is suitable for real-time PCR.
xi. The assay according to embodiment (x), further comprising probes specifically hybridizing to amplification products obtained in said real-time PCR.
xii. The assay according to any of embodiments (x) or (xi), wherein the assay comprises primers and/or probes set forth in the preceding claims.
xiii. The assay according to any of embodiments (x) to (xii), wherein the assay is adapted for use in a fully automated laboratory.
xiv. A diagnostic composition detection of at least nucleic acid of rifampicin and/or isoniazid susceptible and/or resistant Mycobacterium tuberculosis in a biological sample comprising primers and/or probes set forth in any one of the preceding embodiments.
xv. A diagnostic kit detection of at least nucleic acid of rifampicin and/or isoniazid susceptible and/or resistant Mycobacterium tuberculosis in a biological sample comprising primers and/or probes set forth in any one of the preceding embodiments, and optionally comprising instructions for use.
xvi. The diagnostic kit according to embodiments (xv), wherein said kit further comprises enzymes, deoxynucleotides, and/or buffers for performing a reverse transcription step and/or a PCR step.
xvii. The diagnostic kit according to any one of embodiments (xv) or (xvi) further comprising reagents for the isolation of nucleic acids from a biological sample.

### SUMMARY

The invention provides for methods of detecting rifampicin and/or isoniazid susceptible and/or resistant Mycobacterium tuberculosis bacteria in a biological sample using real-time polymerase chain reaction (PCR). Primers and probes for detecting M. tuberculosis are also provided by the invention, as are kits or compositions containing such primers and probes.

Methods of the invention can be used to identify RNA from specimens for diagnosis of Mycobacterium tuberculosis infection. The specific primers and probes of the invention that are used in these methods allow for the amplification and monitoring the development of specific amplification products.

In particular a multi-plex assay for rifampicin and/or isoniazid susceptible and/or resistant Mycobacterium tuberculosis bacteria in a biological sample is provided, which allows for detection and/or diagnosis of bacteria that are susceptible or resistant to these two antibiotics. The primers and probes used in this assay are suitable for the detection of polymorphisms in codons 513, 516, 531 and 533 of the rpoB gene for the detection of polymorphisms in codon 315 of the katG gene as well as for the detection of polymorphisms in the inhA gene promoter region. Polymorphisms in the rpoB gene are associated with alterations in rifampicin resistance in M. tuberculosis. Polymorphisms in the katG gene and in the promoter region of the inhA gene, respectively, are associated with isoniazid resistance.

According to one aspect of the invention, a method for detecting the presence or absence of rifampicin and/or isoniazid susceptible and/or resistant Mycobacterium tuberculosis bacteria in a biological sample from an individual is provided.

The method may comprise a reverse transcription step, at least one cycling step, which includes an amplifying step and a hybridizing step. The amplifying step includes contacting the sample with at least one pair of specific primers to produce an amplification product if a nucleic acid molecule indicative for rifampicin and/or isoniazid susceptible and/or resistant Mycobacterium tuberculosis bacteria is present in the sample. The hybridization step includes contacting the sample with specific probes that are capable of recognizing polymorphisms in the above-mentioned genes rpoB, katG and/or inhA. In the assays of the present invention several primer pairs are used that are suitable to hybridize to respective nucleic acids derived from Mycobacterium tuberculosis, but preferably not to nucleic acids of other bacteria or to nucleic acids encoding other genes. As a result of the methods described herein, the simultaneous amplification and subsequent detection of Mycobacterium tuberculosis that are rifampicin and/or isoniazid susceptible and/or resistant is possible. A pair or set of specific primers comprises a first primer and a second primer. Sequences of the primers and the probes of the invention are shown in the sequence listing.

In some aspects of the invention, the primers and/or probes of the invention can be labeled with a fluorescent moiety. Fluorescent moieties for use in real-time PCR detection are known to persons skilled in the art and are available from various commercial sources, e.g. from life technologies™ or other suppliers of ingredients for real-time PCR.

Representative biological samples from the respiratory tract include throat swabs, throat washings, nasal swabs, and specimens from the lower respiratory tract. In addition, the cycling step can be performed on a control sample. A control sample can include the same nucleic acid molecule that is targeted, katG, rpoB, and inhA. Alternatively, a control sample can include a nucleic acid molecule other than an the target nucleic acid molecule, e.g. a nucleic acid encoding a house-keeping gene or a gene derived from a completely different organism.

Cycling steps can be performed on such a control sample using a pair of control primers and a pair of control probes. The control primers and probes are different from Mycobacterium tuberculosis specific primers and probes.

One or more amplifying steps produce(s) a control amplification product. Each of the control probes hybridizes to the control amplification product.

In another aspect of the invention, there are provided articles of manufacture, or kits.

Kits of the invention can include primer sets specific for the amplification of nucleic acids associated with rifampicin and/or isoniazid susceptibility and/or resistance of Mycobacterium tuberculosis and at least one probe hybridizing specifically with the amplification products. The probe is preferably suitable to specifically hybridize with amplification products obtained with the katG gene-specific primer sets, the rpoB gene specific primer sets and/or the inhA promoter region-specific primer sets of the invention and is suitable to distinguish between amplification products indicative of different polymorphisms in these genes.

Articles of manufacture can include fluorophoric moieties for labeling the primers or probes or the primers and probes are already labeled with donor and corresponding acceptor fluorescent moieties.

The article of manufacture can also include a package insert having instructions thereon for using the primers, probes, and fluorophoric moieties to detect the presence or absence of rifampicin and/or isoniazid susceptible and/or resistant Mycobacterium tuberculosis bacteria in a biological sample.

In another aspect of the invention, there is provided a method for detecting the presence or absence of rifampicin and/or isoniazid susceptible and/or resistant Mycobacterium tuberculosis bacteria in a biological sample from an individual. Such a method includes performing at least one cycling step. A cycling step includes at least one amplifying step and a hybridizing step. Generally, an amplifying step includes contacting the sample with a pair of primers to produce an amplification product if a nucleic acid molecule indicative of rifampicin and/or isoniazid susceptible and/or resistant Mycobacterium tuberculosis bacteria in a biological sample is present in the sample. Generally, a hybridizing step includes contacting the sample with one or more of the specific probes referred to above. The probe is usually labeled with at least one fluorescent moiety. The presence or absence of fluorescence is indicative of the presence or absence of rifampicin and/or isoniazid susceptible and/or resistant Mycobacterium tuberculosis bacteria in a biological sample.

Amplification generally involves the use of a polymerase enzyme. Suitable enzymes are known in the art, e.g. Taq Polymerase, etc.

In another aspect of the invention, there is provided a method for detecting the presence or absence of rifampicin and/or isoniazid susceptible and/or resistant Mycobacterium tuberculosis bacteria in a biological sample from an individual. Such a method includes performing at least one cycling step. A cycling step can include an amplifying step and a dye-binding step. An amplifying step generally includes contacting the sample with a pair of specific primers to produce an amplification product if a target nucleic acid molecule is present in the sample. A dye-binding step generally includes contacting the amplification product with a double-stranded DNA binding dye. The method further includes detecting the presence or absence of binding of the double-stranded DNA binding dye into the amplification product. According to the invention, the presence of binding is typically indicative of the presence of M. tuberculosis nucleic acid in the sample, and the absence of binding is typically indicative of the absence of M. tuberculosis nucleic acid in the sample. Such a method can further include the steps of determining the melting temperature between the amplification product and the double-stranded DNA binding dye. Generally, the melting temperature confirms the presence or absence of M. tuberculosis nucleic acid. Representative double-stranded DNA binding dyes include SYBRGREEN I®, SYBRGOLD®, and ethidium bromide.

In another aspect, the invention allows for the use of the methods described herein to determine whether or not an individual is in need of treatment for M. tuberculosis infection.

Treatment for M. tuberculosis can include, e.g., administration of antibiotics, e.g. antibiotics different from rifampicin and/or isoniazid to the individual.

The invention also provides for the use of the articles of manufacture described herein to determine whether or not an individual is in need of treatment for M. tuberculosis infection.

Further, the methods and/or the articles of manufacture described herein can be used to monitor an individual for the effectiveness of a treatment for M. tuberculosis infection as well as in epidemiology to monitor the transmission and progression of M. tuberculosis infection from individuals to individuals in a population. The methods and/or the articles of manufacture (e.g., kits) disclosed herein can be used to determine whether or not a patient is in need of treatment for M. tuberculosis infection.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### DETAILED DESCRIPTION

According to the present invention, a real-time PCR assay for detecting M. tuberculosis nucleic acid that is more sensitive and specific than existing assays is described herein, in particular for detecting nucleic acids that are indicative of rifampicin and/or isoniazid susceptible and/or resistant Mycobacterium tuberculosis bacteria.

Primers and probes for detecting M. tuberculosis infections, particularly for detecting nucleic acids that are indicative of rifampicin and/or isoniazid susceptible and/or resistant Mycobacterium tuberculosis bacteria, and articles of manufacture containing such primers and probes are also provided. The increased sensitivity of real-time PCR for detection of Mycobacterium tuberculosis as well as the improved features of real-time PCR including sample containment and real-time detection of the amplified product, make feasible the implementation of this technology for routine diagnosis of M. tuberculosis infections in the clinical laboratory, and in particular for detecting nucleic acids that are indicative of rifampicin and/or isoniazid susceptible and/or resistant Mycobacterium tuberculosis bacteria.

The invention provides methods for detecting nucleic acids that are indicative of rifampicin and/or isoniazid susceptible and/or resistant Mycobacterium tuberculosis bacteria by amplifying, for example, a portion of nucleic acid derived from M. tuberculosis genes rpoB, katG or the inhA promoter region and hybridizing the amplification products with specific probes which are preferably fluorescently labeled. Nucleic acid sequences encoding the above genes are available, e.g., in GenBank.

Primers and probes can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length. Designing oligonucleotides to be used as hybridization probes can be performed in a manner similar to the design of primers, although the members of a pair of probes preferably anneal to an amplification product. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 nucleotides in length. Primers useful within the context of the present invention include oligonucleotides suitable in PCR reactions for the amplification of nucleic acids derived from rifampicin and/or isoniazid susceptible and/or resistant Mycobacterium tuberculosis bacteria, respectively.

In describing and claiming the present invention, the terminology and definitions hereinbelow are used for the purpose of describing particular embodiments only, and are not intended to be limiting.

As used in this specification and the appended claims, the singular forms “a,” “an,” and “the” include plural referents unless the context clearly dictates otherwise.

The term “multiplex” refers to multiple assays that are carried out simultaneously, in which detection and analysis steps are generally performed in parallel. As used herein, a multiplex assay may also be an assay that is suitable to simultaneously amplify and identify different target nucleic acids of one particular M. tuberculosis strain.

Within the context of the present invention, a multiplex assay would be for example, a molecular assay that simultaneously screens for nucleic acids that are indicative of rifampicin and/or isoniazid susceptible and/or resistant Mycobacterium tuberculosis bacteria.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

As used herein, the term “target amplification” refers to enzyme -mediated procedures that are capable of producing billions of copies of nucleic acid target. Examples of enzyme-mediated target amplification procedures known in the art include PCR.

Within the context of the present invention, the nucleic acid “target” is the nucleic acid sequence that is indicative of rifampicin and/or isoniazid susceptible and/or resistant Mycobacterium tuberculosis bacteria, e.g. rpoB, katG and/or inhA nucleic acid sequences.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.” In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes binds double-stranded DNA and upon excitation emit light; thus as PCR product accumulates, fluorescence increases. In the context of the present invention, the use of TAQMAN® probes is preferred.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of about 90% or more fall within the scope of the present invention provided they are still capable of identifying the polymorphisms in genes responsible for rifampicin and/or isoniazid resistance/susceptibility. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

As used herein, the term “sample” as used in its broadest sense to refer to any biological sample from any human or veterinary subject that may be tested for the presence or absence of nucleic acids that are indicative of rifampicin and/or isoniazid susceptible and/or resistant Mycobacterium tuberculosis bacteria specific nucleic acids, preferably nucleic acids of the rpoB gene, the katG gene and/or the inhA promoter region. The samples may include, without limitation, tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

The amplification primers and detection probes of the present invention are set forth in the sequence listing.

In one aspect of the invention, there is provided a method for detection of nucleic acids that are indicative of rifampicin and/or isoniazid susceptible and/or resistant Mycobacterium tuberculosis bacteria in a sample comprising the steps of obtaining a biological sample from a patient; isolating nucleic acid from the sample; amplifying the nucleic acid, wherein the nucleic acid is amplified and detected with amplification primers and detection probes selected from the group depicted in the sequence listing.

In another aspect of the invention, there is provided a method for detecting nucleic acids that are indicative of rifampicin and/or isoniazid susceptible and/or resistant Mycobacterium tuberculosis bacteria in a sample comprising the steps of obtaining a tissue sample from a patient; extracting nucleic acids from the sample; amplifying the nucleic acid, wherein the RNA is amplified and detected with amplification primers and detection probes as depicted in the sequence listing.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, and nasopharyngeal washes.

In another embodiment of the invention, the assay is a component of a devices that is suitable in fully automated laboratories capable of extracting nucleic acids from a sample (e.g. using the epMotion System of Eppendorf International), optionally capable of reverse transcribing isolated nucleic acids, performing amplification reactions using the assay components described herein and quantitatively and qualitatively detecting nucleic acid targets, e.g. using real-time PCR.

In a further aspect, the present invention relates to a composition comprising any of the above mentioned primers and probes. Preferably, the composition comprises also ingredients, e.g. enzymes, buffers and deoxynucleotides necessary for reverse transcription and/or PCR, preferably for qualitative and/or quantitative RT-PCR. The composition may be stored in the refrigerator in a liquid state or deep-frozen in a suitable medium, or it may be lyophilized and reconstituted before use and which may further comprises detectable probes and/or an internal control.

The present invention further provides a kit comprising the assay of the invention and optionally instructions for use.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains. All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the detection of at least one rifampicin and/or isoniazid Mycobacterium tuberculosis bacterium in a biological sample, wherein the method comprises:
(a) isolating nucleic acids from the biological sample and optionally performing a reverse transcription, and
(b) conducting real-time PCR, wherein primer sets selected from the sequences depicted in SEQ ID NOs: 1 and 2 and/or SEQ ID NOs: 4 and 5 and/or SEQ ID NOs: 7 and 8 or complements thereof or sequences having at least 90 or 95% identity to any of SEQ ID NOs: 1, 2, 4, 5, 7 or 8 or complements thereof are used, and an oligonucleotide probe is used, said probe specifically recognizing nucleotide polymorphisms in at least one of the following genes: the rpoB gene, the katG gene and the inhA gene of Mycobacterium tuberculosis and wherein at least one probe is selected from the sequences depicted in SEQ ID NOs: 3, 6 and 9 or complements or sequences having at least 90, or 95% identity to any of SEQ ID NOs: 3, 6 or 9 or complements thereof.

2. The method according to claim 1, wherein the primers and or probes carry a fluorescent moiety.

3. An in vitro method for the diagnosis of a Mycobacterium tuberculosis infection in a subject comprising performing the method according to claim 1.

4. A method for monitoring the treatment of Mycobacterium tuberculosis infection, said method comprising performing the method according to claim 1 before treatment with at least one drug and during and/or after treatment with said drug.

5. A real-time PCR assay for the simultaneous detection of at least one nucleic acid of rifampicin and/or isoniazid susceptible and/or resistant Mycobacterium tuberculosis in a biological sample comprising primers and probes having oligonucleotide sequences as defined in claim 1 specifically hybridizing to nucleic acids derived from said Mycobacterium tuberculosis.

6. The assay according to claim 5, wherein the assay is adapted for use in a fully automated laboratory.

7. A composition for the detection of at least one nucleic acid of rifampicin and/or isoniazid susceptible and/or resistant Mycobacterium tuberculosis in a biological sample comprising primers and/or probes having oligonucleotide sequences as set forth in claim 1.

8. A kit for the detection of at least one nucleic acid of rifampicin and/or isoniazid susceptible and/or resistant Mycobacterium tuberculosis in a biological sample comprising primers and/or probes having oligonucleotide sequences as set forth in claim 1, further comprising instructions for use.

9. The kit according to claim 8, wherein said kit further comprises enzymes, deoxynucleotides, and/or buffers for performing a reverse transcription step and/or a PCR step.

10. The kit according to claim 8 further comprising reagents for the isolation of nucleic acids from a biological sample.
